(19) United States
(12) Patent Application Publication
(10) Pub. No.: US 2015/0005769 A1
(43) Pub. Date: Jan. 1, 2015

(54) ELECTROSURGICAL INSTRUMENT AND METHOD

(71) Applicant: OLYMPUS WINTER & IBE GMBH, Hamburg (DE)

(72) Inventors: German KLINK, Berlin (DE); Andreas HÖRLLE, Berlin (DE)

(21) Appl. No.: 14/312,389

(22) Filed: Jun. 23, 2014

(30) Foreign Application Priority Data
Jun. 27, 2013 (DE) 102013212521.6

(51) Int. Cl. A61B 18/14

(52) U.S. Cl. CPC A61B 18/1492; A61B 18/1477; A61B 2018/00607
USPC 606/46

(57) ABSTRACT

The invention relates to an electrosurgical instrument and a method for puncturing a wall of a body lumen. The electrosurgical instrument includes an elongate applicator shank which is guided, in a mobile manner, in a guide catheter, a distal applicator electrode and a catheter electrode, arranged on the guide catheter and electrically insulated with respect to the applicator electrode. The method for puncturing a wall of a body lumen includes the steps of inserting a guide catheter, in which an elongate applicator shank is guided in a mobile manner, up to a wall of a body lumen, applying a radiofrequency AC voltage to a distal applicator electrode and a catheter electrode which is arranged on the guide catheter and electrically insulated with respect to the applicator electrode.

The invention relates to an electrosurgical instrument and a method for puncturing a wall of a body lumen.

Electrosurgical instruments of the type set forth at the outset are known in the prior art and generally comprise a guide catheter, which encloses a lumen in which an applicator, which is also referred to as applicator probe or application probe, with an elongate applicator shank is guided in a mobile manner. These electrosurgical instruments are usually inserted into a body lumen along an insertion direction.

Here, an insertion direction is understood to mean a direction in which an electrosurgical instrument is inserted into a body lumen. Due to anatomical conditions and since an electrosurgical element may be configured to be easy to bend, the insertion direction is not necessarily an ideal straight line. Therefore, the feed direction, in which an electrosurgical instrument is substantially advanced, is considered to be the insertion direction. Therefore, the insertion direction also substantially corresponds to a longitudinal axis of the electrosurgical instrument.

Furthermore, a distal applicator electrode, which forms a distal end of the applicator shank and furthermore forms a surface portion of the applicator shank, is usually arranged on the applicator shank. An applicator tip is usually embodied at a distal end of the applicator electrode, which applicator tip in the case of known applicators is embodied e.g. as mechanically cutting/puncturing tip, e.g. with a trocar polish, or provided with a cutting electrode.

Such electrosurgical instruments serve to emit a radiofrequency electric current to surrounding tissue and are also referred to as coagulation or ablation instruments. Monopolar electrosurgical instruments only require one electrode. During application, this one electrode interacts with a large-area return or neutral electrode, which is likewise connected to the body of a patient. For the purposes of a bipolar application, provision is made for applicators with at least two applicator electrodes, preferably a distal and a proximal applicator electrode. A radiofrequency (RF) voltage with different potentials can be applied (in a bipolar manner) to such bipolar coagulation or ablation instruments, as a result of which the tissue surrounding the electrodes is heated to the extent that body-own proteins denature.

By way of example, the fields of use of electrosurgical instruments include bronchoscopy or the treatment of bronchial carcinomas, in which a guide catheter is inserted up to a target location, or in front of a target location, in the body lumen in order to bring the applicator up to, or in front of, this target location. The target location usually lies in body tissue adjacent to the body lumen. By way of example, in bronchoscopy or in the treatment of bronchial carcinomas, this target location lies in the bronchi of the lungs. Therefore, in order to reach the target location with a distal applicator tip, it is usually necessary to penetrate the body tissue adjoining the body lumen. Particularly if the target location lies in the bronchi, the applicator tip must, to this end, often penetrate into a bronchial wall, which may be cartilaginous. Therefore, applicators often have a mechanically cutting/puncturing applicator tip for mechanical puncturing. However, this may be disadvantageous since such a mechanically cutting/puncturing applicator tip may cause, for example, unwanted damage to a guide catheter and/or unwanted injury of body tissue. Furthermore, high puncturing forces or impulses are in part required to let a mechanically cutting/puncturing applicator tip penetrate into a cartilaginous bronchial wall, which may lead to an unwanted change in location of the applicator. Furthermore, electrosurgical instruments are known, in which provision is made at the distal end for, in addition to the distal coagulation or ablation electrode, a cutting electrode with a substantially smaller surface, as is described, for example, in DE 10 2012 220 682.5. However, it may be complicated to produce such electrosurgical instruments.

It is therefore an object of the present invention to specify an electrosurgical instrument and a method for puncturing a wall of a body lumen, which make it easier to bring an applicator to the target location. In particular, it is an object of the present invention to specify a guide catheter and a method for puncturing a wall of a body lumen, which simplify the penetration of an applicator into a bronchial wall.

According to the invention, this object is achieved by an electrosurgical instrument which comprises an elongate applicator shank which is guided, in a mobile manner, in a guide catheter, and a distal applicator electrode and which electrosurgical instrument moreover provides for a catheter electrode, arranged on the guide catheter and electrically insulated with respect to the applicator electrode.

The invention is based on the discovery that the provision of an electrode on the guide catheter, a so-called catheter electrode, renders it possible to use the distal applicator electrode, which in the actual application of the electrosurgical instrument serves for ablation or coagulation, as cutting electrode when penetrating into a wall made of body tissue, in particular into a cartilaginous bronchial wall. In this manner, the penetration of the distal applicator electrode into the body tissue can be simplified and, as a result thereof, the disadvantages of mechanical puncturing can be avoided or reduced. At the same time, it is possible to avoid the provision of a further electrode, namely a cutting electrode, on the applicator. Rather, the distal applicator electrode itself can be used as cutting electrode if a further electrode, used as return or neutral electrode, is present on the guide catheter.

In addition to the distal applicator electrode, the surgical instrument preferably comprises a second, proximal applicator electrode, which is electrically insulated from the distal applicator electrode, preferably by an insulator. The proximal applicator electrode preferably likewise forms a surface portion of the applicator shank.

Furthermore, the electrosurgical instrument preferably comprises a deflection mechanism, which is arranged and embodied to deflect a distal end of the guide catheter in relation to the insertion direction so as to align the distal end toward a target location. The distal applicator electrode can emerge from this distal end of the guide catheter and can be brought into contact with the wall to be punctured, or just in front of this wall, in order to penetrate therein.

In order to activate the cutting function, a bipolar RF cutting voltage, e.g. 2.5 kV, can preferably be applied to the distal applicator electrode and the catheter electrode. In this manner, an electric arc for electrosurgical cutting can be ignited between the distal applicator electrode and the tissue to be punctured, and discharged by the catheter electrode.

In a preferred configuration, provision is made for the distal end of the applicator shank to have a blunt and/or rounded-off embodiment. Preferably, the distal applicator electrode is provided with a blunt, in particular rounded-off distal end, i.e. the distal applicator electrode has a blunt or rounded-off applicator tip. An advantage of this is that unwanted damage or injury can be avoided or reduced when inserting the applicator through a guide catheter, and also when moving the applicator in a body lumen or in the body tissue.

As a result of the provision of a catheter electrode, an applicator electrode with a corresponding blunt embodiment can also serve as cutting electrode. Here, the electric arc required for cutting is ignited between the region of the applicator tip which is closest to the tissue to be punctured and precisely this tissue.

The catheter electrode is preferably directly or indirectly connectable to an RF voltage source. In particular, the catheter electrode is preferably connectable to an RF voltage source by means of an electrical connection element. By way of example, the electrical connection element can be a metal wire. Furthermore, it is preferable for the electrical connection element to be insulated electrically with respect to the surroundings thereof, for example by means of an insulation sleeve.

In a preferred configuration, provision can be made for the electrical connection element to be embodied as a pulling element of a deflection mechanism. Particularly if the electrosurgical instrument has a deflection mechanism for deflecting the distal end of the guide catheter with respect to the insertion direction, it may be preferable to use a pulling element of such a deflection mechanism simultaneously as electrical connection element for the catheter electrode.

Furthermore, a configuration is preferred in which the catheter electrode is connected to a second applicator electrode in such a way that an electrically conductive connection can selectively be established and switched off between the catheter electrode and the second applicator electrode. In this embodiment, a bipolar applicator is provided, in which the second, proximal applicator electrode is electrically conductively connected to the catheter electrode for puncturing tissue and, in the process, a bipolar RF cutting voltage is applied between the distal applicator electrode and the catheter electrode connected to the proximal applicator electrode. The electrical connection between the catheter electrode and the second, proximal applicator electrode can preferably be separated after puncturing in order to apply an RF coagulation or ablation voltage, e.g. 300 V, in the subsequent coagulation or ablation mode, which coagulation or ablation voltage is then only applied between the two applicator electrodes. Such switching between a cutting mode and a coagulation or ablation mode or correspondingly establishing and separating the electrical connection between the catheter electrode and the proximal applicator electrode is preferably brought about on the proximal end of the electrosurgical instrument, in particular on a handle which is arranged on the proximal end of the electrosurgical instrument.

In a preferred configuration of the electrosurgical instrument, the catheter electrode is arranged on the distal end of the guide catheter. Such a configuration with one at the distal end of the guide catheter is preferred in order to bring the catheter electrode into contact with body tissue in the surroundings of the distal applicator electrode.

Here, the catheter electrode is preferably openly accessible toward the distal end. Furthermore, it is preferable for the catheter electrode to extend over at least one circumferential section of the distal end. By way of example, the catheter electrode can have a ring-shaped embodiment. In particular, the catheter electrode is preferably arranged and embodied to be brought into contact with a wall delimiting a body lumen. In particular, it is advantageous for the catheter electrode to be electrically insulated from a lumen enclosed by the guide catheter. Thus, the catheter electrode is preferably insulated toward the inner side of the guide catheter. This insulation is advantageous for ensuring, in particular, insulation between the catheter electrode and the applicator electrode in the lumen of the guide catheter.

Here, provision can preferably also be made for an anchoring mechanism on the guide catheter, as is described, for example, in the parallel application by the applicant “Führungskatheter mit Verankerungsmechanismus und Verfahren zum Einführen eines Führungskatheters [Guide catheter with anchoring mechanism and method for introducing a guide catheter]” with the same filing date (DE 10 2013 212 448.1). Here, the anchoring mechanism can preferably simultaneously serve as catheter electrode.

A further configuration of the electrosurgical instrument provides for the catheter electrode to be formed by an electrically conductive, viscous fluid, or an electrically conductive gel, which is guided in a lumen of the guide catheter in a manner electrically insulated from the applicator electrode. Here, it is particularly preferred for the fluid or the gel to be able to emerge in a distal section of the guide catheter, for example at the distal end of the guide catheter or at a proximal distance from this distal end. Such a gel electrode is advantageous in that it can establish contact to the surrounding body tissue quickly and over a large area, and therefore can serve as return or neutral electrode.

Here, it may be preferable for a sealing element to be provided at the distal end of the guide catheter, which sealing element seals a lumen of the guide catheter with respect to the distal applicator electrode. By way of example, the sealing element can be embodied as membrane seal or lip seal.

A distal section of the guide catheter preferably has at least one, two or more openings, through which the fluid or the gel can emerge from the lumen of the guide catheter. This opening or these openings can be embodied on the distal end of the guide catheter and/or on the sealing element. The opening or openings can also be embodied on a distal section of the guide catheter as radial opening(s), which extend in the radial direction or obliquely thereto.

Preferably, the electrically conductive, viscous fluid or the electrically conductive gel can initially be applied, for example through a lumen in the guide sleeve and through the distal end of the guide sleeve or through one, two or more openings in the distal end of the guide catheter, before the applicator is guided to the distal end of the guide sleeve or before the applicator tip of the applicator emerges from the distal end of the guide sleeve. If the fluid or gel is applied first and the applicator is subsequently guided to the distal end of the guide sleeve or the applicator tip of the applicator is guided out of the distal end of the guide sleeve, the lumen of the guide catheter and the distal end of the guide catheter can be used for applying the fluid or gel in a particularly simple manner.

A further preferred embodiment variant provides for the catheter electrode to be arranged on a proximal end of a distal section of the guide catheter. In this embodiment variant, the catheter electrode is arranged in such a way that it can come to rest on a wall of the body lumen lying opposite the wall to be punctured, or that it contacts such an opposite wall.

Here too, it is preferable for the catheter electrode to be electrically insulated from a lumen enclosed by the guide catheter. Furthermore, provision is preferably made for the distance between the catheter electrode and a distal end of the guide catheter to correspond to at least one diameter of the body lumen in the region of a target location. In particular, it is preferable for the catheter electrode to form at least part of an external circumference of the guide catheter or to form at least part of an external lateral face of the guide catheter. By way of example, the catheter electrode can have a hollow cylinder-shaped embodiment.

Furthermore, it is particularly preferable for an axial extent of the catheter electrode to correspond to at least one external diameter of the catheter electrode. In particular, it is preferable for the axial extent of the catheter electrode to be greater than the external diameter of the catheter electrode; preferably, the axial extent of the catheter electrode corresponds to at least 1.5 times, in particular at least 2 times the external diameter of the catheter electrode.

Such a large-area configuration of the catheter electrode is preferred in order to ensure that, when the distal end of the guide catheter is deflected to a wall delimiting the body lumen in the region of the target location, the catheter electrode comes into secure contact with the opposite wall in order to be able to serve as return or neutral electrode for the RF cutting voltage.

In a further preferred variant, provision is made for the catheter electrode to be arranged on a distal section of the guide catheter and to be embodied as expandable balloon. An embodiment as one-side expandable balloon is particularly preferred. A one-side expandable balloon should be understood to mean a balloon which, in the expanded state, does not have an unchanging radius as seen from a longitudinal axis of the guide sleeve, but rather has a changing radius. Expressed differently, a one-side expandable balloon is preferably embodied coaxially with the guide sleeve in the non-expanded state, but is preferably not aligned coaxially with the guide sleeve in the expanded state. In particular, it is preferable for the guide sleeve in the expanded state of the balloon to be arranged closer to an inner wall region of the expanded balloon than to an inner wall region, lying opposite thereto in the radial direction, of the expanded balloon. The guide sleeve can also rest against an inner wall region of the expanded balloon.

The balloon preferably has an electrically conductive balloon surface which, in the expanded state, can serve as catheter electrode. In a contracted state, the maximum diameter of the balloon or balloon catheter preferably equals, or is only slightly greater than, the external diameter of the guide catheter. In the expanded state, the maximum diameter of the balloon or balloon catheter preferably corresponds to a diameter of the body lumen, in particular of a lumen in the bronchi, which ensures that the tissue, over which the return line should occur, is in contact with the expanded balloon. Furthermore, the balloon can preferably be expanded by means of an expansion medium. The balloon can be cooled or non-cooled. It is preferable, in particular in the case of a cooled configuration of the balloon, for the expansion medium to be guided into the balloon interior via a first lumen and for a second lumen, which is separate from the first lumen and opens into the balloon interior, to serve for returning the expansion medium to the proximal end of the balloon or balloon catheter. Hence, it is possible to generate a circulation of the expansion medium, wherein the expansion medium preferably contains a cooling liquid, e.g. NaCl solution or the like, or is a cooling liquid.

A particular advantage of a one-side expanding balloon lies in the fact that, as a result of the one-side expansion of the balloon, the guide sleeve can, by supporting the one-side expanded balloon on a bronchial wall, be moved laterally against the opposite bronchial wall and that, therefore, bringing the applicator tip to the target location in an accurate manner can be simplified, in particular when providing a lateral opening at the distal end of the guide sleeve.

In accordance with a further aspect of the present invention, the object set forth at the outset is achieved by a method for puncturing a wall of a body lumen, comprising the following steps: inserting a guide catheter, in which an elongate applicator shank is guided in a mobile manner, up to a wall of a body lumen, applying a radiofrequency AC voltage to a distal applicator electrode and a catheter electrode which is arranged on the guide catheter and electrically insulated with respect to the applicator electrode.

A preferred development of the method provides for the following steps: applying an electrically conductive, viscous fluid, or an electrically conductive gel, as catheter electrode, wherein the fluid or gel is guided preferably in a lumen of the guide catheter, electrically insulated from the applicator electrode, and can furthermore preferably emerge through the distal end of the guide catheter or an opening in a distal section of the guide catheter, inserting the applicator shank up to the distal end of the guide catheter. By virtue of initially applying the fluid or gel and subsequently guiding the applicator shank with the applicator tip to the distal end of the guide catheter, it is possible, for example, to use the distal end of the guide catheter both for the emergence of the fluid or gel and for the subsequent emergence of the applicator tip. In this development, the lumen of the guide catheter can also advantageously initially serve to guide the fluid or gel and subsequently to guide the applicator shank.

The method and the developments thereof preferably have features or method steps which make these particularly suitable for being used with an electrosurgical instrument according to the invention and the developments thereof.

In respect of the advantages, embodiment variants and configuration details of the method and the developments thereof, reference is made to the preceding description relating to the corresponding features of the electrosurgical instrument.

Preferred embodiments of the invention are described in an exemplary manner on the basis of the attached figures. In detail:

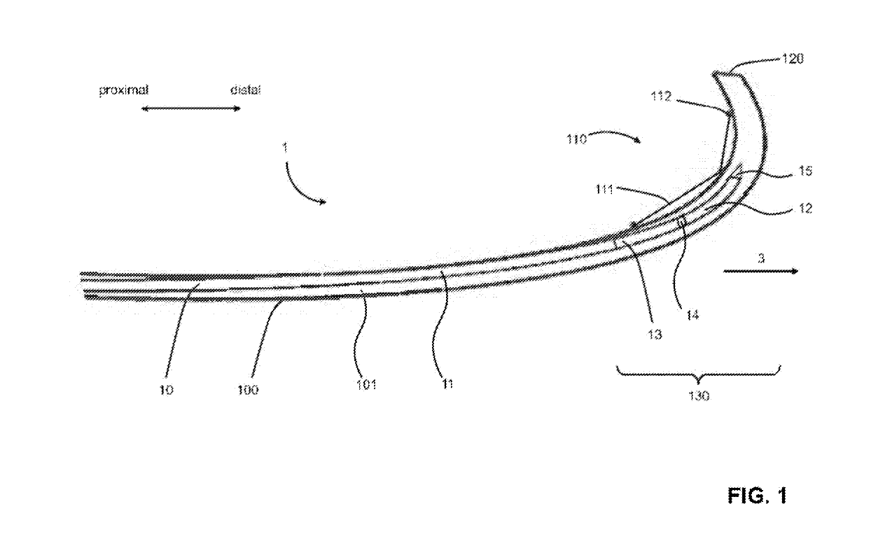

FIG. 1 shows an electrosurgical instrument with a bipolar applicator and a deflection mechanism without catheter electrode;

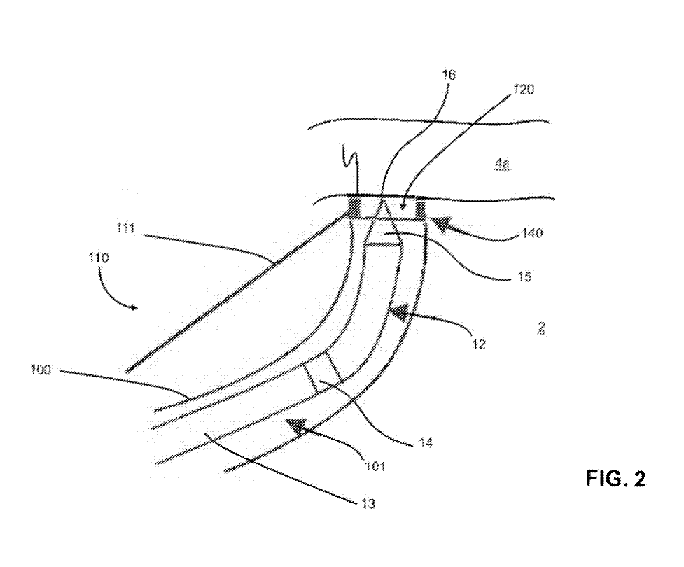

FIG. 2 shows a distal section of an electrosurgical instrument with a bipolar applicator and a catheter electrode arranged at the distal end of the guide catheter;

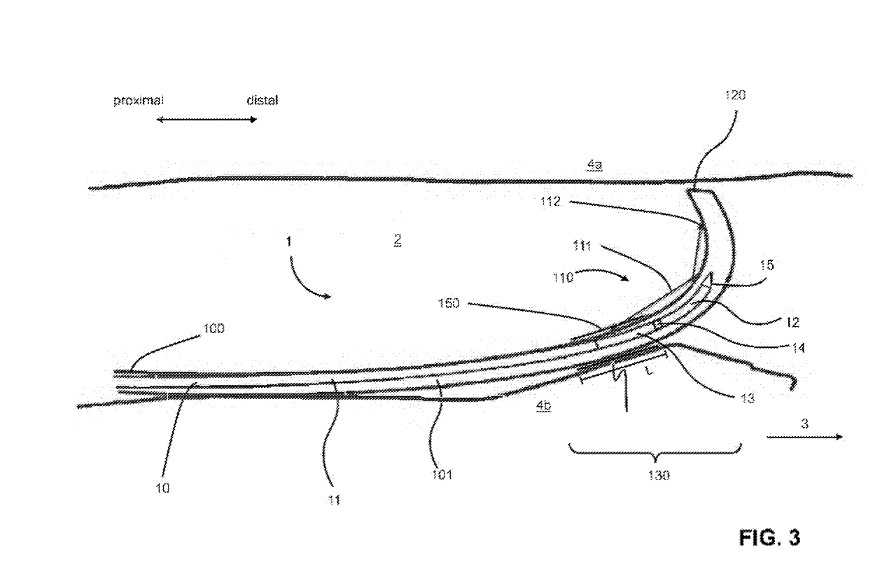

FIG. 3 shows the electrosurgical instrument, depicted in FIG. 1, with a hollow cylinder-shaped catheter electrode;

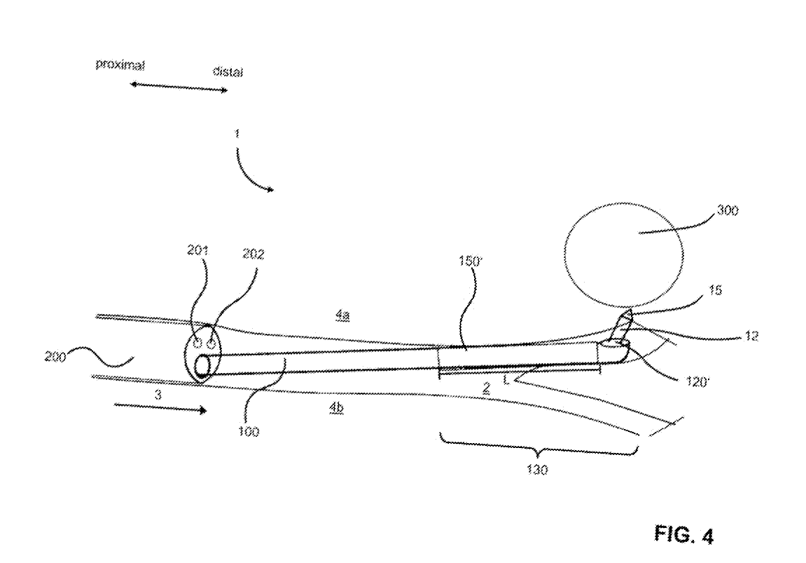

FIG. 4 shows a distal section of an electrosurgical instrument with a further embodiment of a hollow cylinder-shaped catheter electrode;

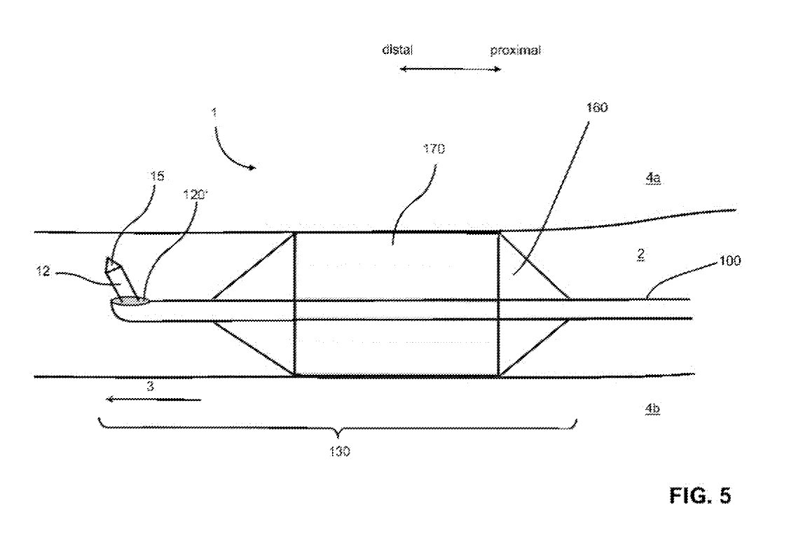

FIG. 5 shows a distal section of an electrosurgical instrument with an applicator and a catheter electrode embodied as a balloon; and

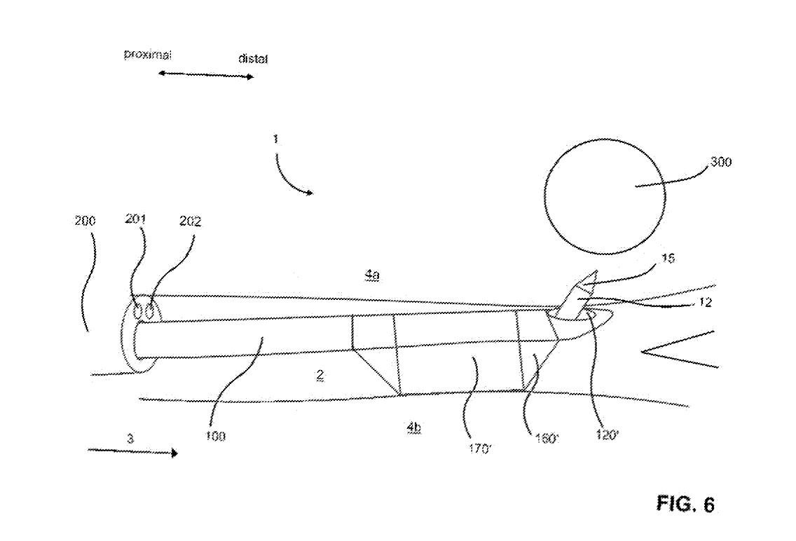

FIG. 6 shows a distal section of an electrosurgical instrument with an applicator and a catheter electrode embodied as a one-side expandable balloon.

FIG. 1 depicts an electrosurgical instrument with a bipolar applicator and deflection mechanism, but without a catheter electrode. FIGS. 2 to 6 each depict an electrosurgical instrument with a catheter electrode, provided according to the invention. The exemplary embodiments in FIGS. 2 and 3 have a deflection mechanism, the exemplary embodiments in FIGS. 4 to 6 have lateral openings in the distal end of the guide sleeve. Equivalent or substantially functionally equivalent elements have been provided with the same reference signs. Unless stated otherwise, the following description relates to all figures.

The figures depict an electrosurgical instrument 1 with a guide catheter for inserting an applicator 10 into a body lumen 2 along an insertion direction 3. The guide catheter comprises a guide sleeve 100, which encloses a lumen 101.

An applicator 10 is arranged in a mobile manner in the lumen 101 of the guide catheter, in particular mobile in the insertion direction 3 relative to the guide catheter. The bipolar applicator 10 has an elongate applicator shank 11 and two applicator electrodes 12, 13 which are arranged successively on the applicator shank 11 in the longitudinal direction of the applicator shank and each form a surface portion of the applicator shank 11. An RF coagulation or ablation voltage can be applied to the applicator electrodes 12, 13, which can serve as coagulation or ablation electrodes (coagulation or ablation mode).

The distal applicator electrode 12 and the proximal applicator electrode 13 are electrically insulated from one another by an insulator 14. The insulator 14 is arranged coaxially with the applicator electrodes 12, 13 and likewise forms a surface portion of the applicator shank 11.

An applicator tip 15 is depicted on the distal applicator electrode 12. Unlike what is depicted in the figures, it is, in particular, also preferable for the distal applicator tip 15 to be embodied in a blunt or rounded-off manner. Overall, a cylinder-shaped design with a substantially constant circular cross section, which tapers at the applicator tip 15, emerges for the applicator shank 11.

The distal end of the guide sleeve 100 preferably has an open embodiment such that a distal end of the applicator 10 with an applicator tip 15 can emerge from the distal end of the guide sleeve 100 so as to be able to advance to a target location in the tissue. In FIGS. 1 to 3, the opening of the distal end 120 is shown as an axial opening. However, as shown in FIGS. 4 to 6, the distal end 120′ can also be embodied in such a way that the opening is aligned laterally or radially.

Furthermore, it may be preferable to provide a sealing element at the distal end of the guide catheter, which sealing element seals a lumen of the guide catheter with respect to the distal applicator electrode. Such a seal is preferred, in particular, in an embodiment (not depicted in the figures) with a catheter electrode embodied as a gel electrode.

Furthermore, the guide catheter can have a deflection mechanism 110, by means of which the distal end 120 of the guide sleeve 100 can be deflected with respect to the insertion direction 3, as depicted in FIGS. 1 to 3. In FIGS. 1 and 2, three connection points 112 can be identified on a distal section 130 of the guide sleeve 100, at which connection points a pulling element, embodied as a pull wire 111, is connected to the distal section 130 of the guide sleeve 100. In FIG. 2, it is only possible to identify the pull wire 111, which is connected to the catheter electrode 140 having a ring-shaped embodiment.

The deflection mechanism 110 depicted in FIGS. 1 to 3 can preferably be tensioned from a proximal end of the guide catheter. To this end, the pull wire 111 can, for example, be guided up to the proximal end of the guide catheter (within, or outside of, the guide sleeve 100). By applying tension to the pull wire 111, the distal end 120 is deflected and aligned in the direction of a bronchial wall 4a (as depicted in FIGS. 2 and 3) and preferably brought into contact with this bronchial wall 4a (as can be identified in FIG. 2).

The applicator 10, in particular the applicator shank 11, preferably has an embodiment that is easy to bend so as to be able to follow a deflection of the distal section 130 of the guide sleeve 100 brought about by the deflection mechanism 110.

The target location, at which a treatment is intended to be performed with the aid of the applicator 10, is preferably situated in the region behind the contact point of the distal end 120, 120′ of the guide sleeve 100 on the bronchial wall 4a, as denoted by 300 in FIGS. 4 and 6.

In the embodiment variant depicted in FIG. 2, the catheter electrode 140 is arranged at the distal end 120 of the guide catheter and has a ring-shaped embodiment. Furthermore, the catheter electrode 140 is electrically insulated from a lumen 101 enclosed by the guide sleeve 100 of the guide catheter. If an RF cutting voltage is now applied to the distal applicator electrode 12 and the catheter electrode 140, an electric arc is ignited at the point 16 of the applicator tip 15 which is closest to the bronchial wall 4a, and discharged via the catheter electrode 140 and the pull wire 111, which serves as electrical connection element. Therefore, the metal wire 111 advantageously serves simultaneously as pulling element for the deflection mechanism 110 and as electrical connection element for the catheter electrode 140. The metal wire 111 is preferably insulated by an insulation sleeve.

In the exemplary embodiment shown in FIG. 3, the catheter electrode 150 with a hollow cylinder-shaped embodiment is arranged at a proximal end of the distal section 13C of the guide catheter. In the embodiment variant shown in FIG. 3, the applicator 10 is to be advanced further into the guide catheter until the applicator tip 15 reaches the bronchial wall 4a. The catheter electrode 150 is also electrically insulated from a lumen 101 enclosed by the guide sleeve 100 of the guide catheter. The distance between the catheter electrode 150 and the distal end 120 of the guide catheter is greater than the distance between the two bronchial walls 4a and 4b delimiting the body lumen 2 in the region of the target location. In the region of the axial extent (L) thereof, the catheter electrode 150 forms the external circumference or the external lateral face of the guide catheter. Furthermore, the axial extent of the catheter electrode is greater than the external diameter of the catheter electrode. As a result of such a large-area configuration, it is possible to ensure that, when the RF cutting voltage is applied on the catheter electrode 150 and the distal applicator electrode 12 (cutting mode), there is contact between the catheter electrode 150 and the opposite bronchial wall 4b.

FIG. 4 depicts the distal end of a bronchoscope 200 with a rinsing channel 201 and an optical channel 202, as well as a guide sleeve 100 in which an applicator is guided. At the distal end 120′ thereof, the guide sleeve 100 has a lateral or radial opening, through which the distal applicator electrode 12 with the applicator tip 15 emerges and, in the example depicted in FIG. 4, it has already penetrated the bronchial wall 4a in order to advance to the target location 300, in this case a bronchial tumor. In the example depicted in FIG. 4, the catheter electrode is embodied as a hollow cylinder-shaped catheter electrode 150′, which is arranged at a proximal end of a distal section 130 of the guide catheter. In the example shown in FIG. 4, the hollow cylinder-shaped catheter electrode 150′ has a length L corresponding to a multiple of the external diameter of the catheter electrode 150′. In the example shown in FIG. 4, the catheter electrode 150′ extends almost over the whole distal section, denoted by 130, of the guide sleeve 100, i.e. almost to the distal end 120′ of the guide sleeve 100 with the lateral or radial opening. Such a configuration is preferred, in particular, for bronchial channels with a small diameter.

FIGS. 5 and 6 show exemplary embodiments, in which the catheter electrode is embodied as an expandable balloon 160, 160′. FIGS. 5 and 6 show the balloon 160, 160′, which has an electrically conductive balloon surface 170, 170′, in the expanded state. The balloon 160, 160′ can be embodied in a cooled or non-cooled manner. In the depicted expanded state, the maximum diameter of the balloon catheter 160, 160′ preferably corresponds substantially to the diameter of the lumen between the bronchial walls 4a,b, which ensures that the bronchial tissue 4a,b, via which the return feed is intended to be brought about, is in contact with the electrically conductive surface 170, 170′ of the expanded balloon 160, 160′. The distal end 120′ of the guide catheter is embodied as a lateral or radial opening in the examples shown in FIGS. 5 and 6. However, the opening can also be embodied as an axial opening.

The embodiment variant depicted in FIG. 6 shows a one-side expandable balloon 160′ with an electrically conductive surface 170′. In FIG. 6, it is possible to identify that the one-side expandable balloon 160′, depicted here in the expanded state, supports the guide sleeve 100 on the bronchial wall 4b and, as a result thereof, moves the distal end 120′ with a lateral or radial opening in the direction of the opposite bronchial wall 4a. Therefore, the distal end 120′ with a lateral opening is brought to rest on the bronchial wall 4a. As a result, the one-side expandable balloon 160′ can ensure not only the function of a catheter electrode for discharging the cutting voltage in the cutting mode by means of the electrically conductive balloon surface 170′, but can simultaneously support or simplify the alignment of the distal end 120′ with respect to the target location 300 and the support of the distal end 120′.

In all variants of the figures, an RF cutting voltage can be applied to the distal applicator electrode 12 and the catheter electrodes 140, 150, 150′, or the catheter electrodes embodied as a balloon 160, 160′, such that an electric arc is ignited between the point of the applicator tip 15 which is closest to the bronchial wall 4a and the bronchial wall 4a or 4b so as to cut into the possibly cartilaginous bronchial wall 4a and therefore simplify the penetration of the distal end of the applicator 10 into the bronchial wall 4a. Once such a cut in the bronchial wall 4a is performed and the distal end of the applicator electrode 12 has as a result penetrated into the bronchial wall 4a, it is possible to change from a cutting mode to an ablation or coagulation mode, for the purposes of which an RF coagulation or ablation voltage is applied to the applicator electrodes 12, 13.

Therefore, by providing a catheter electrode, the distal applicator electrode can be used not only as coagulation or ablation electrode, but also as cutting electrode for simplifying the penetration into a wall delimiting a body lumen.

### LIST OF REFERENCE SIGNS

1 Electrosurgical instrument
2 Body lumen
3 Insertion direction
4a Bronchial wall
4b Opposite bronchial wall
10 Applicator
11 Applicator shank
12 Distal applicator electrode
13 Proximal applicator electrode
14 Insulator
15 Applicator tip
16 Point on the applicator tip closest to the bronchial wall
100 Guide sleeve
101 Lumen
110 Deflection mechanism
111 Pulling element embodied as pull wire
112 Connection points
120, 120′ Distal end of the guide sleeve
130 Distal section of the guide sleeve
140 Catheter electrode with a ring-shaped embodiment
150, 150′ Catheter electrode with a hollow cylinder-shaped embodiment
160, 160′ Expandable balloon
170, 170′ Electrically conductive balloon surface
200 Bronchoscope
201 Rinsing channel
202 Optical channel
300 Target location, tumor
L Length of the catheter electrode

1. Electrosurgical instrument, comprising
an elongate applicator shank which is guided, in a mobile manner, in a guide catheter,
a distal applicator electrode,
a catheter electrode, arranged on the guide catheter and electrically insulated with respect to the applicator electrode.

2. Electrosurgical instrument according to claim 1,
wherein the distal end of the applicator shank has a blunt and/or rounded-off embodiment.

3. Electrosurgical instrument according to claim 1,
wherein the catheter electrode is connected to a second applicator electrode in such a way that an electrically conductive connection can selectively be established and switched off between the catheter electrode and the second applicator electrode.

4. Electrosurgical instrument according to claim 1,
wherein the catheter electrode is arranged on the distal end of the guide catheter.

5. Electrosurgical instrument according to claim 1,
wherein the catheter electrode extends over at least one circumferential section of the distal end of the guide catheter.

6. Electrosurgical instrument according to claim 1,
wherein the catheter electrode has a ring-shaped embodiment.

7. Electrosurgical instrument according to claim 1,
wherein the catheter electrode is electrically insulated from a lumen enclosed by the guide catheter.

8. Electrosurgical instrument according to claim 1,
wherein the catheter electrode is formed by an electrically conductive, viscous fluid, or an electrically conductive gel, which is guided in a lumen of the guide catheter in a manner electrically insulated from the applicator electrode.

9. Electrosurgical instrument according to claim 1,
wherein a sealing element is provided at the distal end of the guide catheter, which sealing element seals a lumen of the guide catheter with respect to the distal applicator electrode.

10. Electrosurgical instrument according to claim 8,
wherein a distal section of the guide catheter has an opening through which the fluid or the gel can emerge.

11. Electrosurgical instrument according to claim 1,
wherein the catheter electrode is arranged on a proximal end of a distal section of the guide catheter.

12. Electrosurgical instrument according to claim 11,
wherein the distance between the catheter electrode and a distal end of the guide catheter corresponds to at least one diameter of the body lumen in the region of a target location.

13. Electrosurgical instrument according to claim 11,
wherein the catheter electrode forms at least part of an external circumference of the guide catheter.

14. Electrosurgical instrument according to claim 11,
wherein an axial extent of the catheter electrode corresponds to at least one external diameter of the catheter electrode.

15. Electrosurgical instrument according to claim 1,
wherein the catheter electrode is arranged on a distal section of the guide catheter and embodied as expandable balloon.

16. Method for puncturing a wall of a body lumen comprising the following steps:
inserting a guide catheter, in which an elongate applicator shank is guided in a mobile manner, up to a wall of a body lumen,
applying a radiofrequency AC voltage to a distal applicator electrode and a catheter electrode which is arranged on the guide catheter and electrically insulated with respect to the applicator electrode.

17. Method according to claim 16, comprising the following steps:
applying an electrically conductive, viscous fluid, or an electrically conductive gel, as catheter electrode,
inserting the applicator shank up to the distal end of the guide catheter.
